(19) United States
(12) Patent Application Publication
(10) Pub. No.: US 2006/0275243 A1
(43) Pub. Date: Dec. 7, 2006

(54) Cosmetic or therapeutic combination preparation with theanine

(75) Inventors: Gabriele Blume, Strasse (DE); Dirk Teichmuller, Linsengericht (DE)

Correspondence Address: SIMPSON & SIMPSON, PLLC, 5555 MAIN STREET, WILLIAMSVILLE, NY, 14221-5406, US

(73) Assignee: ROVI GmbH & Co. Kosmetische Rohstoffe KG, Schluchtern (DE)

(21) Appl. No.: 11/437,954

(22) Filed: May 19, 2006

(30) Foreign Application Priority Data
May 25, 2005 (DE) DE 102005024040.2

(51) Int. Cl. A61K 8/55; A61K 8/97; A61K 31/198; A61K 36/02

(52) U.S. Cl. 424/70.21; 424/74; 424/195.17; 514/561; 424/70.23

(57) ABSTRACT

A cosmetic or therapeutic composition with a proportion of substantially free amino acids as well as the use of such a composition for improving skin moistness, skin elasticity and/or for skin wrinkle reduction. In accordance with the invention proposed for that purpose is a cosmetic or therapeutic composition having a proportion of substantially free amino acids, which includes at least theanine and an amino acid selected from aspartic acid and glutamic acid, wherein the proportion of substantially free amino acids additionally contains at least two amino acids which are selected from alanine, glycine, threonine and serine. The invention also includes a method for preparing the composition.

### BACKGROUND OF THE INVENTION

The invention concerns a cosmetic or therapeutic combination preparation (composition) with a proportion of substantially free amino acids. In addition the invention concerns the use of such a combination preparation for the production of a cosmetic for improving skin moistness, skin elasticity and/or for skin wrinkle reduction.

The human skin forms a barrier between the environment and the interior of the body. It will be appreciated that at the same time that barrier is also a contact surface, that is to say the skin is also constantly subjected to demands and stresses due to its contact with the environment. Those demands can be both physical (radiation, mechanical loading) and also chemical (contact with liquids such as for example soapsuds) or biological (bacteria, fungus, insect bites or stings). Those environmentally induced stresses generally result in the natural ageing process of the human skin being speeded up.

In the search for suitable means which can slow down, stop and/or in the best-case scenario even reverse the skin ageing processes, the primary aim is generally that of finding an agent which in particular reduces, avoids or reverses the external effect of the ageing process, above all wrinkle formation. Various approaches have already been followed hitherto in order to achieve that.

The best-known and oldest principle of anti-wrinkle agents is applying suitable greases. So-called re-greasing preparations are intended to preclude wrinkle formation by feeding the skin with greases which have been removed therefrom by the everyday demands, for example by frequently washing the hands. A further principle is that of the so-called moisturising creams. They are intended primarily to positively influence the moisture balance of the skin as it is assumed that skin ageing correlates in particular with inadequate skin moisture.

There is no doubt that the above-indicated products are effective to a certain extent but do not afford the desired result in a sufficiently sustained fashion. Rather, when using those products, it is generally necessary for them to be applied at least once daily. The complaint is also frequently made that, after concluding application of those products, the skin very quickly returns to the same state as prior to the treatment or in part even a worse state than previously. In other words, in many cases it is not really possible to refer to slowing down the skin ageing processes and in particular restoring the skin wrinkles which have already been formed, but rather what is involved is only temporary wrinkle smoothing. Thus when using moisturising creams and re-greasing preparations a satisfactory state can usually only be attained if the products are applied daily and in some cases even several times a day.

Further some anti-wrinkle agents which are available on the market act by accelerating removal of hard skin surface, in part even causing direct skin detachment (peeling) and at the same time stimulating re-formation of the treated skin. Fruit acids are frequently incorporated into suitable cosmetic preparations for that purpose. Those fruit acids however cannot be readily applied in relation to every type of skin and many cases are known in which the use of those agents resulted in skin irritation.

Therefore conventional anti-wrinkle agents generally do not afford the desired wrinkle smoothing operation and certainly do not do so while at the same time effecting a slowing down of the ageing processes of the skin while reducing numbers and severity of skin wrinkles which have already formed. Further such conventional anti-wrinkle agents frequently result in unpleasant and sometimes even painful skin irritation.

There is therefore a need for an anti-wrinkle agent which has the object of overcoming the disadvantages of the above-mentioned conventional preparations and which therefore in particular affords a sustainable improvement in skin moistness as well as a sustainable increase in skin elasticity and skin strength without for that purpose the need for an excessively frequent, such as for example daily, application. Further a desired object is that an anti-wrinkle agent be found that meets the above requirements while permitting stopping of application, even over prolonged periods of time (some days), without the state of the skin markedly worsening again.

### BRIEF DESCRIPTION OF THE INVENTION

In accordance with the invention the above objects are attained by a composition having cosmetic and therapeutic properties which composition includes a proportion of substantially free amino acids, wherein the proportion of substantially free amino acids contains at least theanine and an amino acid which is selected from aspartic acid and glutamic acid, and wherein the proportion of substantially free amino acids additionally contains at least two amino acids which are selected from alanine, glycine, threonine and serine. The free amino acids in the above composition preferably contain at least 80 weight percent theanine.

### BRIEF DESCRIPTION OF THE DRAWINGS

FIG. 1A is a graph showing the results of an investigation of moisture-providing properties of a control gel over time as determined in Example 2.

FIG. 1B is a bar graph showing the results of an investigation of moisture providing properties of the control gel shown in FIG. 1A with free amino acids in accordance with the invention, as determined in Example 2.

FIG. 2 shows a bar graph comparing the results of an investigation of the elasticity-improving properties of a combination preparation as set forth in Example 2 in comparison with a base control gel.

### DETAILED DESCRIPTION OF THE INVENTION

For the composition according to the invention, it was possible to demonstrate that it already significantly improves the skin moistness and the elasticity of the skin in the treated region after a two-week treatment. In addition after two weeks' treatment it was possible to see in the treated region a markedly perceptible reduction in the number of fine wrinkles in the skin. In addition it was also possible to perceive a marked smoothing effect for the corresponding parts of the skin. Furthermore it was possible to establish that the positive effects which are achieved by use of the preparation according to the invention persist for several days and can still be seen even a week after termination of the treatment. Further details regarding those positive properties of the combination preparation according to the invention will be apparent from the examples set forth hereinafter.

A large part of the skin wrinkles occurs solely due to reflex movements of the parts of the skin including the tissue layers therebeneath. That gives rise for example to wrinkles on the forehead as well as laughter wrinkles. Stress conditions in the skin which can lead to the formation even of relatively small wrinkles are however also triggered off by a strong (in particular) wind, draft or also by precipitations such as for example rain. In order to counteract nervous stressing of parts of the skin which are loaded in that way, the amino acid theanine is added to the combination preparation according to the invention. Theanine is an amino acid which is characteristic of the tea plant Camellia sinensis, in the leaves of which it is contained in high proportions in the form of a free amino acid. That amino acid is attributed the property of being able to release physic tensions. Thus investigations have shown for example that theanine can inhibit the cramp-triggering effect of caffeine.

It will be noted however that the positive effects of theanine can presumably only reduce wrinkle formation and not compensate for the entire complex of symptoms involved in skin ageing. Therefore further additives were sought for a new agent for the preventative and therapeutic treatment of the ageing processes of the human skin. It was assumed for that search that in the living world there should be some organisms whose external sealing tissue, by virtue of their way of life, is particularly heavily stressed. A habitat in which such organisms were suspected to occur appeared inter alia to be the tidal zone of the seas, as it is here that the ebb and flow of the tides on the one hand usually gives rise to a high level of mechanical loading on the sealing layers of the organisms which are living there, and in addition there is a constant change between dry and moist periods. In addition the organism should also be in a position to endure substantial fluctuations in temperature in its environment.

One region which satisfied all the above-mentioned demands is the tidal zone of the Atlantic coast of Namibia. That is inter alia the habitat of the green alga Enteromorpha intestinalis occurring sessile on stones and rocks. That alga is on the one hand adapted to life in heavily salt-bearing water but on the other hand it is also highly resistant in relation to long dry periods (during the period of the tidal ebb) and in relation to substantial fluctuations in temperature (temperatures between 2 and 30° C.).

It is assumed that the resistance of that alga is due to given biomolecules (for example proteins) which can compensate for the high stresses involved. In order to find that out, proteins which are characteristic of that alga were extracted from the alga and enzymatically hydrolysed. The prepared extract obtained in that way (referred to hereinafter as alga hydrolysate) is rich in natural amino acids and contains no salts. In particular the following free amino acids were found in the alga hydrolysate, in the proportions specified in parentheses, with respect to the total amino acid content of the hydrolysate (=100%): aspartic acid (13-18% by weight), glutamic acid (2-17% by weight), alanine (8-12% by weight) and a proportion of 6-9% by weight of each of threonine, glycine and/or serine respectively.

It was now found that this amino acid combination is suitable as a basic composition for a novel anti-wrinkle agent. For example the specific amino acid cocktail established from Enteromorpha intestinalis is suitable on the one hand for cranking up cellular metabolism and on the other hand improving the state of cell and tissue support structures such as for example the fibril-forming structural proteins collagen, elastin and keratin which are also referred to as scleroproteins.

It is striking for example that the above-mentioned alga hydrolysate contains very high proportions of aspartic acid and glutamic acid. It is known of those two amino acids that they decisively intervene in metabolism of the cells. Thus aspartic acid is involved in the conversion of carbohydrates into energy so that a deficiency in aspartic acid can lead to a reduction in the energy present in a cell. Glutamic acid is also involved in carbohydrate metabolism and in addition also acts on the fat metabolism and the synthesis processes for DNA, glutathione and other amino acids. In addition glutamic acid and aspartic acid are essential constituents of collagen. It is therefore proposed that at least one of those two amino acids must be contained in the preparation according to the invention.

Furthermore the alga hydrolysate also has a considerable proportion of further collagen components. Thus the amino acids alanine and glycine are represented in large proportions, that is to say the amino acids which alone make up 36% of a collagen polymer. So-to-speak glycine and alanine are also the essential constituents of the scleroprotein elastin in which they make up about 50% of the polymer structure. In regard to the scleroprotein keratin also the above-mentioned alga hydrolysate affords a large part of the essential constituents. Serine contained in the alga hydrolysate, glutamic acid and aspartic acid are also included among those amino acids. In addition the alga hydrolysate also contains considerable proportions of the amino acid threonine. That amino acid is an essential amino acid which cannot be synthesised by the human body so that it must usually be absorbed by way of nutrition. That amino acid is also an essential component of scleroprotein and in particular is also of significance as a constituent of the cell membrane.

It is assumed that the supply of scleroprotein constituents (possibly in excess) causes a shift in the equilibrium of synthesis/breakdown of scleroproteins towards synthesis. It is however also possible that the provision of those amino acids will only compensate for a system-inherent chronic deficiency of those constituents. At any event it is necessary for the combination preparation according to the invention to contain at least two of the structural constituents alanine, glycine, threonine and serine. Which of the four amino acids available are used depends primarily on which scleroprotein is to be formed to an intensified degree and to what extent also the cell membranes are to be strengthened. The combination of alanine and glycine presents itself for example if it is primarily collagen and/or elastin formation or maintenance that is to be promoted. The addition of serine is aimed at keratin promotion and threonine is generally used for scleroprotein promotion and for cell membrane optimisation.

An embodiment which has proven to be a particularly advantageous composition for a combination preparation according to the invention is one in which the component of substantially free amino acids includes the amino acids aspartic acid, glutamic acid, glycine, threonine, alanine and serine. In a particularly preferred feature those amino acids are used in the form of an alga hydrolysate which was preferably produced from the extract of a green alga. In other words, in a preferred embodiment of the present invention, the amino acids are added in the form of such an alga hydrolysate with the amino acid composition particular to that alga hydrolysate, to the preparation. In a particularly preferred feature for that purpose a hydrolysate of a green alga of the genus Enteromorpha is used. In particular the hydrolysate of the green alga Enteromorpha intestinalis is preferred.

Preferably the proportion of the substantially free amino acids with respect to the finished preparation is 0.5 to 10% by weight. In specific embodiments that proportion is 3 to 6% by weight and proportions of substantially free amino acids in the finished combination preparation of about 5% by weight are particularly preferred. With respect to that overall proportion of the substantially free amino acids in the preparation, the proportion of theanine is preferably 80-95% by weight. Theanine proportions in the range of 85-95% by weight are particularly preferred and in particular theanine proportions of 90 to 94% by weight are preferred. The proportion in the combination preparation which is made up by the substantially free amino acids aspartic acid, glutamic acid, glycine, threonine, alanine and serine is preferably 5 to 20% by weight with respect to the total proportion of the substantially free amino acids in the preparation. Preferably that proportion is 5 to 15% by weight and in a particularly preferred feature the total of the proportions of the above-mentioned amino acids, with respect to the total proportion of the substantially free amino acids in the preparation, is 6 to 10% by weight.

In particularly preferred embodiments, with respect to the total proportion of the substantially free amino acids in the preparation, which are different from theanine, the aspartic acid content is 13 to 18% by weight. In further embodiments the glutamic acid content is 12 to 17% by weight and in alternative embodiments the alanine content is 8 to 12% by weight. It is particularly preferred if all of the three above-mentioned amino acids are contained in the combination preparation in the specified ranges of proportion. It is further preferred if with respect to the total proportion of the substantially free amino acids in the preparation, the proportions of threonine, glycine and/or serine are each 6 to 9% by weight respectively.

As many green alga hydrolysates, besides the above-mentioned amino acids, also have considerable proportions of proline, leucine and/or phenylalanine, specific preferred embodiments of the invention, with respect to the total proportion of the substantially free amino acids in the preparation, which are different from theanine, also contain 5 to 8% by weight of each of proline, leucine and/or phenylalanine. In particular proportions of proline and/or leucine are preferred as it is assumed that those amino acids, as significant constituents of scleroproteins, can also be responsible for a further improvement in the effect of the preparation according to the invention. The proportion of proline in collagen is about 20% and in elastin it is about 12%. Leucine is contained in a proportion of about 4% in collagen and 12% in elastin. Proline and leucine also contribute to a relatively great extent to the structure of the molecule in keratin.

The term substantially free amino acids is used here to denote those amino acids which are not a fixed constituent of a peptide and which preferably are present in unbonded form. The term substantially free amino acids however is also used to denote those amino acids which are bonded to other substances by way of hydrogen bridge bonds or ionic bonds or van der Waals forces as well as those amino acids which are reversibly covalently bonded to carrier substances and which are liberated from the carrier substances at the latest at the location of action, for example by hydrolytic cleaving of the bond. In specific embodiments however the substantially free amino acids are already at least partially bonded in the preparation to a carrier substance or to various carrier substances. The only decisive consideration in that respect is that the amino acids are finally readily present in their originally free form at the location of action, that is to say in the skin or in the cells.

As the amino acids which are necessarily and/or preferably contained in the combination preparation according to the invention are relatively up to strongly polar, it is preferred that the combination preparation additionally includes a cosmetically compatible carrier which optimises transport through the upper skin layers and through the cell membranes into the cells so that the bioavailability of the effective substances is as high as possible. Preferably such a carrier includes membrane-forming lipids. It is particularly preferred if those membrane-forming lipids are present in vesicular form. Suitable for that purpose are in particular the membrane-forming lipids which are selected from the groups of phospholipids, ceramides and diazylglycosides. Also preferred are combination preparations with membrane-forming lipids which include mixtures of substances from the aforementioned groups.

In particularly preferred embodiments of the present invention the membrane-forming lipids contain at least 70% by weight of phosphatidyl choline and particularly preferably about 80 to 90% by weight of phosphatidyl choline. A further preferred embodiment is wherein the carrier additionally contains linoleic acid in stabilised form. Preferably the combination preparation contains the stabilised linoleic acid in an amount of 3.5 to 6.5% by weight.

In addition the combination preparation preferably includes at least one cosmetically compatible additive. The additives which are usually employed for cosmetic or therapeutic preparations fall to be considered as additives here. In preferred embodiments methylparabene, sorbic acid and/or potassium dihydrogen phosphate are contained as additive in the combination preparation.

As already mentioned the combination preparations according to the invention have outstanding effects on the ageing processes of the human skin. Therefore the effective, substantially free amino acids contained in a combination preparation according to the invention, namely theanine and an amino acid which is selected from aspartic acid and glutamic acid, and additionally at least two amino acids which are selected from alanine, glycine, threonine and serine, are used for the production of a cosmetic for improving skin moistness, skin elasticity and/or skin wrinkle reduction.

It is particularly preferred for the amino acid combination according to the invention to be produced at least partially, that is to say excluding at least theanine, from alga material, for example from a protein extract of the green alga Enteromorpha intestinalis.

The production of the proteins from the alga is effected in accordance with methods which are known to the man skilled in the art. Preferably acid hydrolysis is not used for breaking down the proteins obtained from the alga, as in that case upon subsequent neutralisation of the hydrolysate produced, large amounts of salt occur. Thus upon acid hydrolysis with hydrochloric acid for example large amounts of common salt occur, which are unwanted in the preparation and which can only be removed with difficult from the amino acid mixture. Therefore in a preferred method of producing a combination preparation according to the invention the alga proteins are enzymatically broken down with proteases into the individual amino acids thereof That method also prevents chemical reactions and conversions of the amino acids which can occur at a low pH-value which is produced by the addition of acid in the acid hydrolysis operation.

A particularly preferred combination preparation according to the invention is therefore in particular wherein the alga hydrolysate contained in the preparation or the hydrolysate of the alga extract contained in the preparation is substantially free from salts.

In a particularly preferred method of producing a combination preparation according to the invention firstly the substantially free amino acids are dissolved in water in suitable amounts until clear with agitation at a maximum of 40° C. That solution is then combined with an ethanolic lethicin solution and the two solutions are slowly brought together with turraxing (=homogenisation using a TURRAX® homogeniser) and then brought to a vesicle diameter size of about 100 to 250 nm by high-pressure homogenisation, extrusion and/or mechanical reduction in size in some other fashion. Aqueous phosphate buffer is then added with steady homogenisation and homogenisation is further continued until the result is a slightly viscous homogeneous emulsion. The pH-value of the emulsion is adjusted if required with conventional means to about pH 5.5 to 7.0. That generally produces a yellowish-brown fluid which has a slight ethanol odour and a characteristic lethicin odour.

The combination preparation according to the invention is preferably incorporated into a cosmetic or pharmaceutical carrier matrix, particularly preferably in a use concentration of 1.0 to 5.0% by weight. The carrier matrix can be gel formulations, cream formulations (O/W and W/O emulsions), lotions, mask applications and so forth.

A method of formulating a combination preparation according to the present invention in the form of a gel is to be described in the following manner: a thickener, preferably in an amount of 0.1 to 3.0% by weight, and a non-ionic emulsifier, preferably in an amount of 1.0 to 15.0% by weight, and in a particularly preferred embodiment, a co-emulsifier, are completely dissolved in water with slight agitation. At a maximum of 30° C., one of the above-described embodiments of the combination preparation according to the invention is homogeneously stirred into that matrix, preferably in an amount of 1.0 to 5.0% by weight. Finally a preserving agent can be added, preferably in an amount of 0.1 to 0.5% by weight, and further homogeneously stirred in. The gel exhibits a clear to cloudy appearance. The viscosity varies in dependence on the nature and concentration of the thickener used. The pH-value of the gel is adjusted if necessary to about 5.5 to 7.0 with conventional means.

It is pointed out that all features as are disclosed to a man skilled in the art from the present description, the Figures and the claims can be combined both individually and also in any combinations with others of the features or groups of features disclosed here insofar as that has not been expressly excluded or such combinations would be technically impossible or meaningless. That applies even if the individual features or groups of features have been described specifically only in connection with certain other features. A detailed presentation of all conceivable combinations of features is dispensed with here only for the sake of brevity and readability of the description. By way of example however some of some combinations of features and further advantages, features and possible uses of the present invention will be apparent from the following Examples and the associated Figures.

### EXAMPLE 1

A composition (combination preparation) according to the invention is produced in accordance with the above-specified method and includes the following constituents:
16.0% by weight Ethanol, non-denatured
10.0% by weight Phospholipids (lecithin/PL80)
5.0% by weight L-theanine (99%)
0.5% by weight Alga hydrolysate
(15.8% aspartic acid
14.7% glutamic acid
10.5% alanine
10.5% glycine
7.4% leucine
7.3% threonine
6.9% serine
6.2% proline
5.1% phenylalanine)
0.05% by weight Methylparaben
0.03% by weight Sorbic acid
0.5% by weight Potassium dihydrogen phosphate
ad 100% by weight Water

Firstly the free amino acid L-theanine and the free amino acids of the alga hydrolysate were completely dissolved in water at 40° C. The result is a clear light-brown solution of a pH-value of 5.8 to 6.2. An ethanolic lethicin solution which had been produced at about 50° C. was slowly added to the aqueous amino acid solution with turraxing (=homogenisation at 3,000 to 10,000 rpm) and then extruded through a 200 nm polycarbonate filter. Finally the phosphate buffer was added with steady homogenisation and homogenisation continued until a low-viscosity homogeneous emulsion was produced. The vesicle size expressed as the diameter of the liposome hollow balls was determined with a ZETAMASTER S from Malvern Instruments, UK, using the method of photon correlation spectroscopy (PCS) at 152 nm. If the desired pH-value were not attained directly if necessary it would be possible to set it to a pH-value of 5.5 to 7.0 with NaOH solution.

### EXAMPLE 2

The composition according to the invention as set forth in Example 1 was incorporated into a gel formulation in a concentration of use of 5.0% by weight. The gel matrix used here was carbomer gel. Incorporation was effected with agitation at a temperature of 30° C.

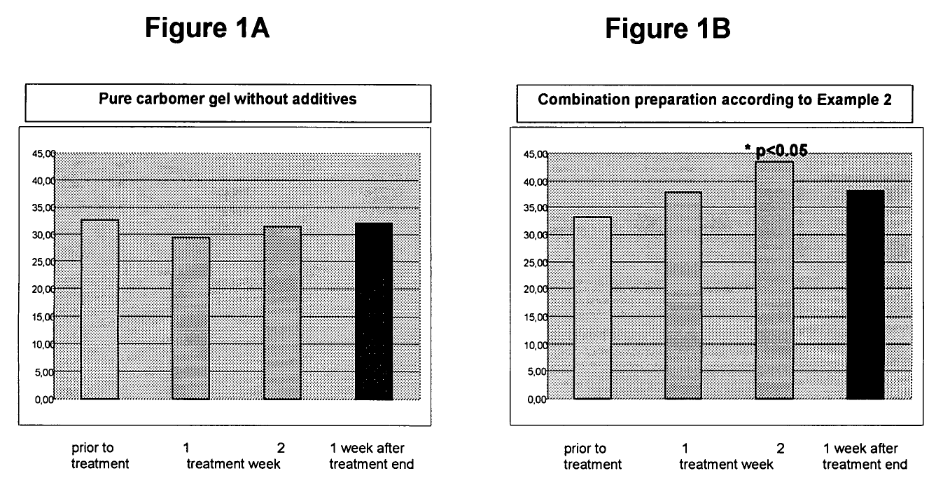

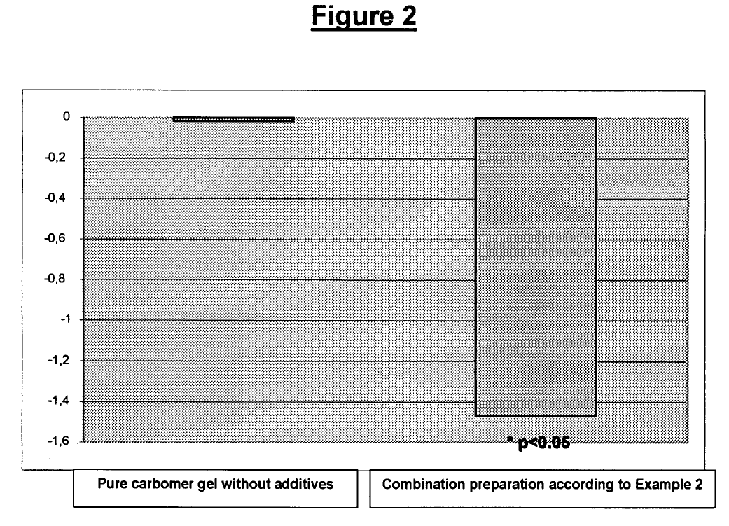

The result of use of that preferred composition of the composition according to the invention is shown in following FIGS. 1A, 1B and 2.

FIGS. 1A and 1B show the results of an investigation of the moisture-dispensing properties of a combination preparation as set forth in Example 2.

FIG. 2 shows the results of an investigation of the elasticity-improving properties of a combination preparation as set forth in Example 2 as compared with a base control gel.

A composition according to the invention as set forth in Example 2 was tested on six volunteer subjects over a period of two weeks for the study, the results of which are shown in FIGS. 1A, 1B and 2. The gel formulation was applied twice daily in regions with fine wrinkles, namely around the eyes and on the inside of the forearm. Prior to treatment, during the two-week period of the treatment and a week after the treatment, the following parameters were determined: 1. skin moistness (FIGS. 1A and 1B), 2. skin elasticity (FIG. 2) and 3. the number and depth of the wrinkles (not shown). To determine the skin moistness the treated regions at the inner forearm were determined by means of a corneometer. The graph in FIG. 1A shows the measured values (relative magnitudes) of the regions which were treated with pure carbomer gel (without active substance). FIG. 1B shows the results of the skin regions which were treated with a combination preparation according to the invention as set forth in Example 2. The higher the values plotted on the ordinate, the correspondingly higher also is the level of skin moistness. It can be seen that the treatment with pure carbomer gel does not cause a significant change, not to mention an improvement in the moistness of the parts of the skin which were treated with the gel.

It can also be seen from the graph in FIG. 1B that after just a week of treatment with combination preparation according to the invention, a marked improvement in skin moistness is to be found. That improvement markedly increases once again in the second week. The value which was measured one week after termination of the treatment indicates that, even at that time, there was still a markedly better level of skin moistness than prior to the treatment. It is to be judged therefrom that the action of the combination preparation according to the invention not only persists for a short time, that is to say only one or a few days, but that the action still lasts even after a week. This test demonstrates the great advantage that the combination preparation according to the invention has over the conventional agents, namely that it is markedly more sustained in its action. In other words, in order to arrive at the desired state, the user does not have to apply the preparation daily but even a use at regular intervals of for example three or four days or even application once a week affords a marked improvement over the state prior to the treatment.

FIG. 2 shows the result of the elasticity test in which the region with fine wrinkles in the proximity of the eye was measured with a cutometer. The measurement after a two-week treatment shows that the stress state of the skin, by treatment with a combination preparation according to the present invention, stands out clearly from the treatment with the matrix gel without the addition of amino acids according to the invention. More specifically the lower the value (relative magnitude) which was achieved with that test, the correspondingly better is the increase in skin elasticity achieved.

The reduction in the number and depth of the fine wrinkles by a two-week treatment with the combination preparation according to the invention was evaluated optically. In that procedure it was possible to find an objective improvement over the treatment with the pure gel matrix. In other words the treatment with the combination preparation according to the invention resulted in a visible reduction in the number of wrinkles and at the same time also provided a reduction in wrinkle depth (skin-smoothing action) of the wrinkles which could still be seen.

What is claimed is:

1. A composition having cosmetic and therapeutic properties comprising a combination of substantially free amino acids, wherein the combination of substantially free amino acids contains at least theanine and at least one amino acid which is selected from aspartic acid and glutamic acid, and wherein the proportion of substantially free amino acids additionally contains at least two amino acids which are selected from alanine, glycine, threonine and serine.

2. A composition according to claim 1 wherein the proportion of substantially free amino acids includes the amino acids aspartic acid, glutamic acid, glycine, threonine, alanine and serine.

3. A composition according to claim 1 wherein it includes the hydrolysate of an alga or of an extract of an alga.

4. A composition of claim 3 where the alga a green alga.

5. A composition according to claim 4 where the green alga is a green alga of the genus Enteromorpha.

6. A composition of claim 5 where the green algae is Enteromorpha intestinalis.

7. A composition according to claim 3 wherein the hydrolysate is substantially free from salts.

8. A composition according to claim 1 wherein the composition contains from about 0.5 to about 10% by weight of finished composition.

9. The composition of claim 8 wherein the composition contains from about 3 to about 6% by weight of finished composition.

10. A composition according to claim 1 wherein theanine is present in an amount of from about 80 to about 95% by weight of essentially free amino acids in the composition.

11. A composition according to claim 10 wherein theanine is present in an amount of from about 85 to about 95% by weight of essentially free amino acids in the composition.

12. A composition according to claim 1 wherein theanine is present in an amount of from about 90 to about 94% by weight of essentially free amino acids in the composition

13. A composition according to claim 1 wherein the proportion of the substantially free amino acids which are different from theanine, with respect to the total proportion of the substantially free amino acids other than theanine in the composition includes
a) 13 to 18% by weight of aspartic acid,
b) 12 to 17% by weight of glutamic acid, and
c) 8 to 12% by weight of alanine, and
d) 6 to 9% by weight of each of threonine, glycine and serine.

14. A composition according to claim 1 wherein the preparation includes a cosmetically compatible carrier.

15. A composition according to claim 14 wherein the carrier includes membrane-forming lipids.

16. The composition of claim 15 wherein the membrane-forming lipids are present in vesicular form.

17. A composition according to claim 15 wherein the membrane-forming lipids are selected from phospholipids, ceramides, diacylglycosides and mixtures thereof.

18. A composition according to claim 17 wherein the membrane-forming lipids contain at least 70% by weight of phosphatidyl choline.

19. A composition according to claim 15 wherein the carrier further includes linoleic acid in stabilised form in an amount of 3.5 to 6.5% by weight.

20. A composition according to claim 1 wherein it includes at least one cosmetically compatible additive which is selected from methylparaben, sorbic acid and potassium dihydrogen phosphate.

21. A composition according to claim 1 wherein the substantially free amino acids in the composition are present bonded to at least one carrier substance selected to liberate the amino acids after application of the composition to skin.

22. A method for treating skin to improve moisture, increase elasticity and/or reduce wrinkles comprising applying a composition according to claim 1 to the skin.

23. A method for treating skin to improve moisture, increase elasticity and/or reduce wrinkles comprising applying a composition according to claim 10 to the skin.

24. A method for the preparation of a composition for treatment of skin to improve moisture, increase elasticity and/or reduce wrinkles comprising mixing substantially free amino acids with a cosmetically compatible carrier wherein the substantially free amino acids include the amino acid theanine, at least one of the amino acids aspartic acid or glutamic acid, and at least two amino acids which are selected from alanine, glycine, threonine and serine.

25. A method according to claim 24 where the theanine is at least 80 percent by weight of essentially free amino acids in the composition

26. A method according to claim 25 wherein at least a portion of the amino acids are obtained by enzymatic cleaving of alga proteins.

27. The method of claim 26 where the alga protein is a protein of green algae of the genus Enteromorpha.
